(19) United States
(12) Patent Application Publication
(10) Pub. No.: US 2020/0121173 A1
(43) Pub. Date: Apr. 23, 2020

(54) OVERTUBE DEVICE

(71) Applicant: OLYMPUS CORPORATION, Tokyo (JP)

(72) Inventor: Masaru YANAGIHARA, Tokyo (JP)

(73) Assignee: OLYMPUS CORPORATION, Tokyo (JP)

(21) Appl. No.: 16/721,282

(22) Filed: Dec. 19, 2019

Related U.S. Application Data
(63) Continuation of application No. PCT/JP2017/024191, filed on Jun. 30, 2017.

(51) Int. Cl. A61B 1/01; A61B 1/00; A61B 1/005

(52) U.S. Cl. CPC A61B 1/01; A61B 1/00052; A61B 1/00071; A61B 1/0051; A61B 1/00154

(57) ABSTRACT

An overtube device includes: a tube body having a bending part and a main body part; a wire having a distal end part fixed to the bending part and a proximal end part positioned on the proximal end side and extending along a longitudinal axis; an operation part capable of pulling the wire to the proximal end side; and an overtube base having a stopper that fixes the tube body in a longitudinal axis direction so as not to advance and retract, and engages with the operation part in a state where the wire is pulled toward the proximal end side of the wire so as to hold a curved shape of the bending part. When a medical device inserted into the tube body is retracted, an engagement between the operation part and the stopper is released, and holding of the curved shape of the bending part is released.

### CROSS REFERENCE TO RELATED APPLICATIONS

This application is a continuation application based on a PCT Patent Application No. PCT/JP2017/024191, filed on Jun. 30, 2017, the content of which is incorporated herein by reference.

### BACKGROUND

### Technical Field

The present invention relates to an overtube device.

### Background Art

Conventionally, an overtube for assisting a procedure for inserting a medical device such as an endoscope or a treatment tool into a body cavity or a lumen, for example, a deep part of a large intestine or a small intestine is known. The overtube is flexible and has a lumen (channel, conduit) through which a medical device such as an endoscope or a treatment instrument can be inserted.

The insertion part of the medical device is inserted through the lumen of the overtube, and is inserted into the body cavity or the lumen together with the overtube. Further, when the overtube is inserted into the body cavity or lumen first, the insertion part of the medical device is inserted along the lumen of the overtube.

Thus, the overtube functions as a guide for the insertion part of the medical device. As a result, even when the body cavity or lumen has a bending part, the insertion part of the medical device can be smoothly inserted into the body cavity or deep part of the lumen.

The insertion part of the medical device is inserted through the lumen of the overtube, and the distal end part protrudes from the distal end of the overtube.

When the medical device is a treatment instrument, a treatment part such as a grip provided at the distal end of the treatment instrument protrudes from the distal end of the overtube. The affected part is treated by the treatment part protruding from the distal end of the overtube.

Japanese Unexamined Patent Application, First Publication No. 2009-279412 (hereinafter referred to as Patent Document 1) discloses an overtube with a shape lock function. A part of the overtube described in Patent Document 1 includes a plurality of telescopic elements. By pulling the wires inserted through the plurality of telescopic elements toward the proximal end in the longitudinal axis direction of the overtube, the intimate force between the telescopic elements is increased. As a result, the shape of the overtube is temporarily fixed by the frictional force generated between the telescopic elements.

By the overtube whose shape of the overtube is temporarily fixed, medical devices such as an endoscope and a treatment instrument can be arranged stably when treating an affected part in a flexible body cavity or lumen. Moreover, by the overtube whose shape is temporarily fixed, it is possible to more reliably guide the distal end of the treatment instrument to the distal end of the overtube.

### SUMMARY

An overtube device includes: a tube body having a bending part that is curvable on a distal end side, and a main body part that is continuous with the bending part and extends to a proximal end side; a wire having a distal end part fixed to the bending part, and a proximal end part positioned on the proximal end side of the main body part and extending along a longitudinal axis of the main body part; an operation part that is provided on the proximal end side of the main body, is attached to the proximal end part of the wire, and is configured to be capable of pulling the wire to the proximal end side of the wire; and an overtube base having a stopper configured to fix the tube body in a longitudinal axis direction so as not to advance and retract, and to engage with the operation part in a state where the wire is pulled toward the proximal end side of the wire so as to hold a curved shape of the bending part. When a medical device inserted into the tube body is retracted, an engagement between the operation part and the stopper is released, and holding of the curved shape of the bending part is also released at the time when the medical device is retracted.

The operation part may include: an operation part main body; and a wire operation part that is held so as to be relatively movable with respect to the operation part main body and to which the proximal end part of the wire is attached. By moving the wire operation part relative to the operation part main body, the wire may be pulled toward the proximal end side of the wire.

The operation part main body may include an advance/retreat stopper engaging part. The wire operation part may include a wire stopper engaging part. The stopper of the overtube base may include an advance/retreat stopper configured to engage with the advance/retreat stopper engaging part, and a wire stopper configured to engage with the wire operating part while keeping a relative position with the advance/retreat stopper constant. The advance/retreat stopper may engage with the advance/retreat stopper engaging part and the wire stopper may engage with the wire stopper engaging part, so that the operation part is mounted on the overtube base.

The wire stopper may be configured to be movable to either a first position or a second position. When the wire stopper that engages with the wire stopper engaging part is disposed at the first position, the operation part may pull the wire toward the proximal end side of the wire, and the curved shape of the bending part may be held. When the wire stopper that engages with the wire stopper engaging part is disposed at the second position, the operation part may not pull the wire toward the proximal end part of the wire, and the curved shape of the bending part may be released.

The overtube base may include: a sensor configured to detect a position of a proximal end of the medical device; a stopper drive part configured to operate the stopper; and a controller configured to determine whether or not the medical device is retracted based on a detection result by the sensor, and to operate the stopper drive part so that the engagement between the operation part and the stopper is released when it is determined that the medical device is retracted. By release of the engagement between the operation part and the stopper by the stopper drive part, holding of the curved shape of the bending part may be released when the medical device is retracted.

The overtube base may include: a sensor configured to detect a position of a proximal end of the medical device; a stopper drive part configured to operate the stopper; and a processor configured to determine whether or not the medical device is retracted based on a detection result by the sensor, and to generate a command to operate the stopper drive part so that the engagement between the operation part and the stopper is released when it is determined that the medical device is retracted. By release of the engagement between the operation part and the stopper by the stopper drive part based on the command from the processor, holding of the curved shape of the bending part may be released when the medical device is retracted.

The processor may control an operation of bending a distal end part of the medical device. When it is determined that the medical device is retracted and when a curved shape of the distal end part is a shape that contacts the tube body, the processor may control the engagement between the operation part and the stopper to release the holding of the curved shape of the bending part.

The overtube base may include: a retraction detection part; and a link that connects the stopper to the retreat detection part. A proximal end part of the medical device may engage with the retraction detection part and the stopper may be operated in conjunction, so that the engagement between the operation part and the stopper is released.

The wire may be a plurality of wires, and the stopper of the overtube base engages with the operation part in a state where the plurality of wires may be simultaneously pulled toward a proximal end side of the plurality of wires.

### BRIEF DESCRIPTION OF THE DRAWINGS

FIG. 1 is a diagram showing an overall configuration of an overtube device according to an embodiment of the present invention.

FIG. 2 is a side view of the overtube device.

FIG. 3 is a cross-sectional view of a flexible tube part of the overtube device.

FIG. 4 is a cross-sectional view of a bending piece of the overtube device.

FIG. 5 is a cross-sectional view of the overtube in which the shape lock function of the overtube device is invalidated.

FIG. 6 is a cross-sectional view of the overtube in which the shape lock function of the overtube device is activated.

FIG. 7 is a cross-sectional view when the wire stopper of the overtube device is disposed at a first position.

FIG. 8 is a cross-sectional view when the wire stopper of the overtube device is disposed at a second position.

FIG. 9 is a diagram showing the operation of the overtube device.

FIG. 10 is a diagram showing the operation of the overtube device.

FIG. 11 is a diagram showing the operation of the overtube device.

FIG. 12 is a diagram showing the operation of the overtube device.

FIG. 13 is a diagram showing the operation of the overtube device.

FIG. 14 is a diagram showing the operation of the overtube device.

FIG. 15 is a diagram showing a preparation procedure for the overtube device.

FIG. 16 is a control flowchart of the controller of the overtube device.

FIG. 17 is a cross-sectional view of a modification of the overtube device.

FIGS. 18A and 18B are cross-sectional views of a modification of the overtube device.

### DETAILED DESCRIPTION OF THE PREFERRED EMBODIMENTS

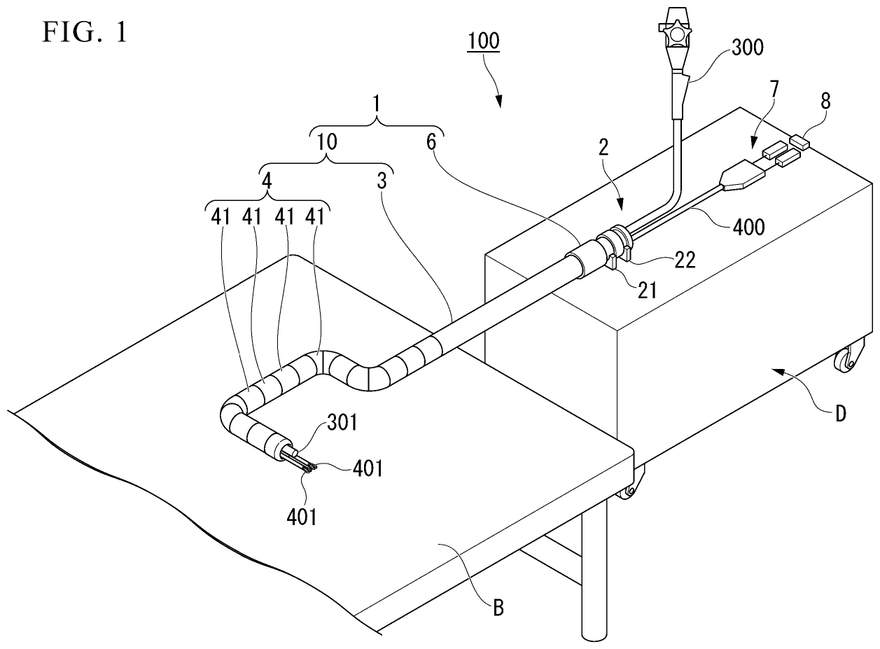

A first embodiment of the present invention will be described with reference to FIGS. 1 to 18B. In addition, in order to make the drawings easy to see, the thicknesses and dimensional ratios of the respective constituent elements are appropriately adjusted.

FIG. 1 is a diagram showing an overall configuration of an overtube device 100 according to the present embodiment.

The overtube device 100 includes an overtube 1 and an overtube base 2.

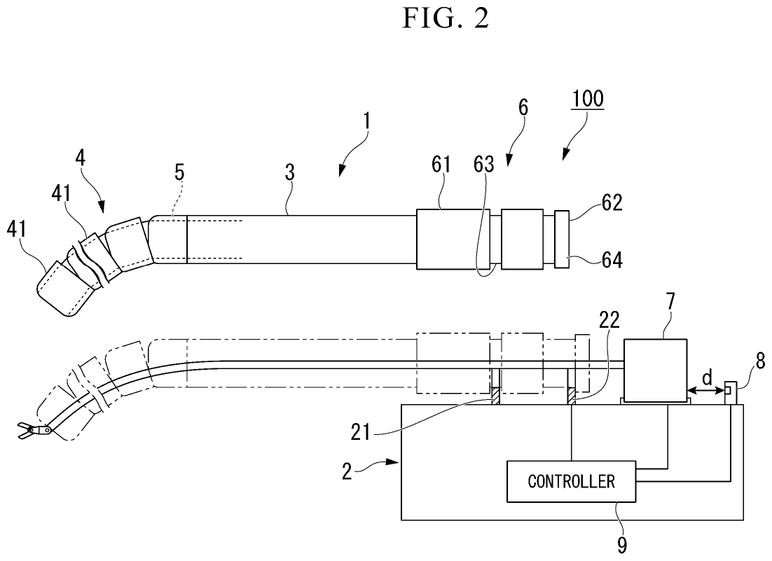

FIG. 2 is a side view of the overtube device 100. As shown in FIG. 2, an operation part 6 is detachably attached to the overtube base 2.

As shown in FIGS. 1 and 2, the overtube 1 includes a tube body 10, a wire 5, and the operation part 6 provided at the proximal end of a flexible tube part 3. The tube body 10 has a bending part 4 that can be bent on the distal end side, and has the flexible tube part (main body part) 3 that extends to the proximal end side in connection with the bending part 4.

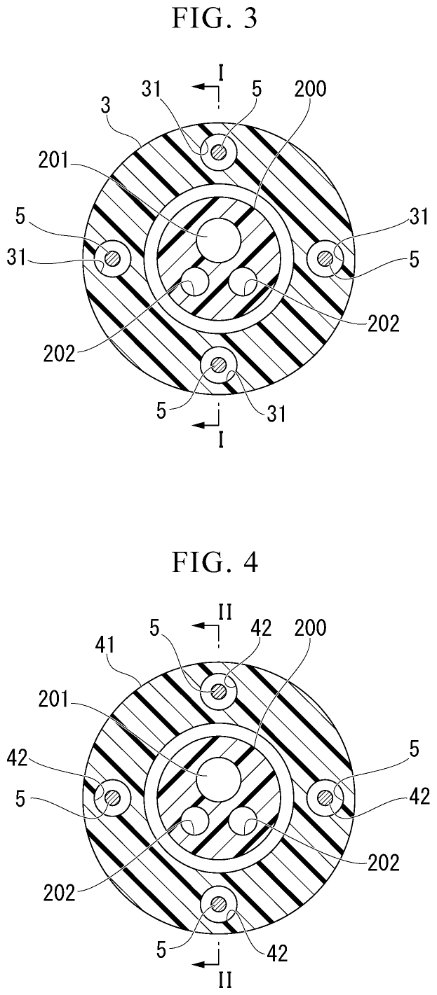

FIG. 3 is a cross-sectional view of the flexible tube part 3.

The flexible tube part 3 is a tubular member formed of a flexible material such as silicone, for example, and a multi-lumen tube 200 described later is inserted through the inside thereof as shown in FIG. 3.

As shown in FIG. 3, the flexible tube part 3 is provided with four wire lumens 31 through which the wire 5 for temporarily fixing (shape locking) the shape of the bending part 4 described later is inserted. As shown in FIG. 4, the four wire lumens 31 are arranged at positions that equally divide the circumference around the longitudinal axis of the flexible tube part 3.

As shown in FIGS. 1 and 2, the bending part 4 is configured by arranging a plurality of bending pieces 41 arranged in the axial direction, and is provided at the distal end of the flexible tube part 3.

The bending piece 41 is a short cylindrical member, and the internal space is open at both ends. The plurality of bending pieces 41 are overlapped so that the internal space of the adjacent bending pieces 41 is a continuous space. A multi-lumen tube 200 described later is inserted through the continuous internal space.

FIG. 4 is a cross-sectional view of the bending piece 41.

As shown in FIG. 4, all the bending pieces 41 are provided with four wire lumens 42 in the same manner as the wire lumen 31 provided in the flexible tube part 3. As shown in FIG. 4, the four wire lumens 42 are arranged at positions that equally divide the circumference around the longitudinal axis of the bending part 4.

The wire 5 has a distal end part fixed to the bending part 4 and a proximal end part located on the proximal end side of the flexible tube part 3, and extends along the longitudinal axis of the flexible tube part 3. The wire 5 is inserted through all the bending pieces 41 and the wire lumens (31, 42) of the flexible tube part 3. The distal end of the wire 5 is attached to the bending piece 41 on the most distal side.

Since the wires 5 are inserted through all the bending pieces 41, the bending pieces 41 are not separated from each other. By moving the bending piece 41 relative to the adjacent bending piece 41, the entire bending part 4 can be bent. However, when the wire 5 inserted through the inside of the bending part 4 is not loose, the bending part 4 cannot be further bent from the current curved shape.

The wire 5 is pulled toward the proximal end side of the wire 5, the bending pieces 41 are in close contact with each other, and a frictional resistance occurs between the bending pieces 41, thereby the curved shape of the bending part 4 is held. In a case where at least two wires 5 are provided, when a plurality of wires 5 are simultaneously pulled toward the proximal end side of the wires 5, the bending pieces 41 come into close contact with each other, and friction resistance occurs between the bending pieces 41, thereby the curved shape of the bending part 4 is held. In the following description, holding of the curved shape of the bending part 4 by the overtube base 2 to be described later in a state where the wire 5 (the plurality of wires 5) is pulled to the proximal end side of the wire 5 until the curved shape of the bending part 4 is held is referred to as “activating the shape lock function” of the bending part 4. Moreover, releasing the holding of the curved shape of the bending part 4 by releasing the pulling of the wire 5 by the overtube base 2 to be described later is referred to as “disabling the shape lock function”. The means for pulling the wire 5 to the proximal end side of the wire 5 may be electric pulling or manual pulling.

As shown in FIG. 2, the bending piece 41 on the distal end side in the longitudinal axis direction of the bending part 4 is dome shaped. By the bending part 4 configured by such bending pieces 41, the contact part between adjacent bending pieces 41 can be increased as much as possible when the bending piece 41 is relatively moved so that the entire bending part 4 is bent. By increasing the contact part between the adjacent bending pieces 41, the frictional resistance acting between the bending pieces 41 can be increased, and the bending part 4 can more suitably perform the shape lock function.

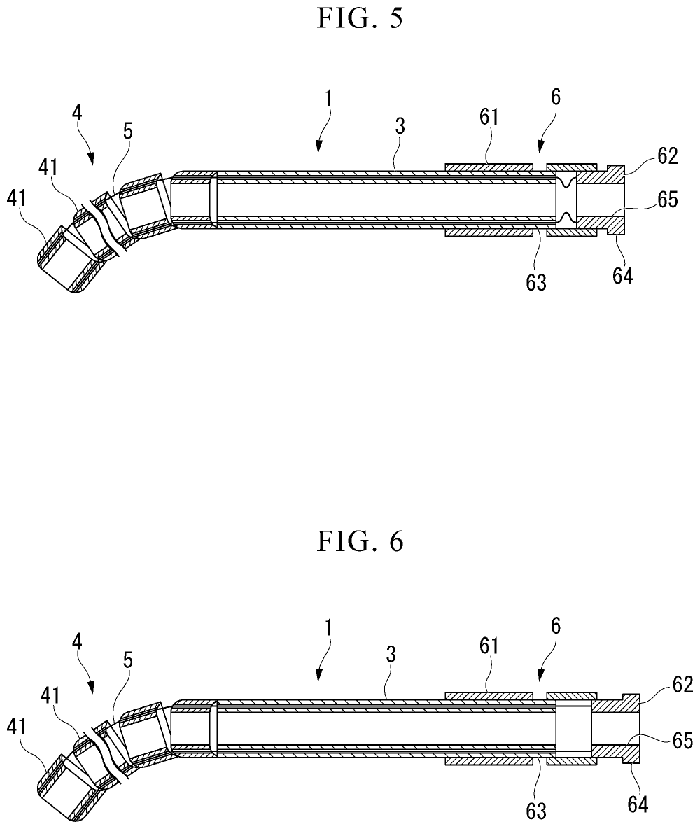

FIGS. 5 and 6 are diagrams for explaining the drive mechanism of the shape lock function of the overtube 1, and are sectional views taken along the line I-I in FIG. 3 and the line II-II in FIG. 4. Note that the description of the multi-lumen tube 200 is omitted.

FIG. 5 is a cross-sectional view of the overtube 1 in which the shape lock function is invalidated. On the other hand, FIG. 6 is a cross-sectional view of the overtube 1 in which the shape lock function is activated.

As shown in FIG. 5, in the overtube 1 in which the shape lock function is invalidated, the wire 5 is loose. Therefore, the bending part 4 can be bent until the wire 5 is not loosened.

On the other hand, as shown in FIG. 6, the wire 5 is not loose in the overtube 1 in which the shape lock function is activated. Therefore, it is limited to further bend the bending part 4 from the current curved shape. That is, when the operation part 6 is mounted (attached) to the overtube described later in a state where the wires 5 (the plurality of wires 5) are pulled simultaneously to the proximal end side of the wires 5 until the curved shape of the bending part 4 is held, the wire 5 is held in a state of being pulled toward the proximal end side. As a result, the curved shape of the bending part 4 is held.

As shown in FIGS. 5 and 6, the operation part 6 includes an operation part main body 61 to which the proximal end of the flexible tube part 3 is connected, and a wire operation part 62 provided slidably with respect to the operation part main body 61.

The operation part main body 61 is a tubular member made of a material having high rigidity. As shown in FIG. 2, its outer diameter is larger than the outer diameter of the flexible tube part 3, and it is formed in a shape that is easy for an operator to grasp.

As shown in FIG. 2, a concave advance/retract stopper engaging part 63 that engages with an advance/retreat stopper 21 of the overtube base 2 described later is formed on the outer periphery of the operation part main body 61.

As shown in FIGS. 5 and 6, the internal space of the operation part main body 61 communicates with the internal space of the flexible tube part 3.

As shown in FIGS. 5 and 6, the wire operation part 62 is a tubular member having an outer diameter smaller than the inner diameter of the operation part main body 61, and is held so as to be relatively movable in the longitudinal axis direction with respect to the operation part main body 61. Further, the proximal end part of the wire 5 is attached to the wire operation part 62. When the operator moves the wire operation part 62 relative to the operation part main body 61 toward the proximal end side, the wire operation part 62 pulls the wire 5 to increase the tension of the wire 5. By increasing the frictional force of the portion where the bending pieces 41 contact with each other and holding the curved shape, the shape lock function of the bending part 4 can be activated.

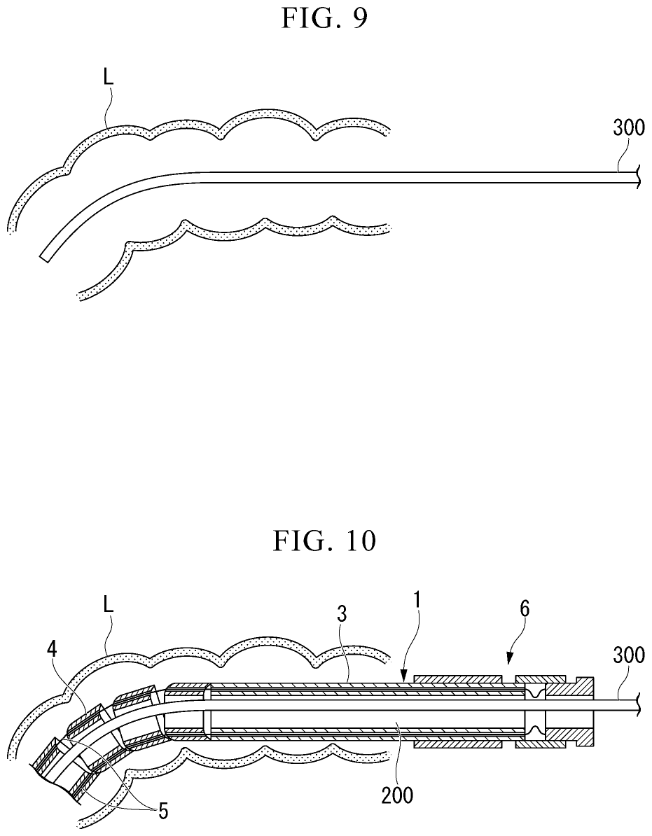

On the proximal end side of the wire operation part 62, a wire stopper engaging part 64 that engages with a wire stopper 22 of the overtube base 2 described later is formed. As shown in FIGS. 6 and 9, the wire stopper engaging part 64 is a convex portion having an outer diameter larger than the outer diameter of the other portion of the wire operation part 62.

As shown in FIGS. 5 and 6, the proximal end of the wire operation part 62 is open, and the multi-lumen tube 200 is inserted through the opening 65. The inserted multi-lumen tube 200 passes through the internal space of the operation part main body 61, the flexible tube part 3, and the bending part 4, and protrudes from the distal end of the bending part 4.

The length of the wire 5 is adjusted to an appropriate length so that, even when the bending part 4 is in a straight state without being bent, the wire operation part 62 is not separated from the operation part main body 61.

The overtube base 2 is a base on which the operation part 6 can be detachably mounted. In the present embodiment, the overtube base 2 is integrated with a carriage D with casters, as shown in FIG. 1. The carriage D may be an integrated operation console including an operation input part that can operate the treatment instrument 400 or the like.

When using the overtube device 100, the casters of the carriage D are fixed so as not to move. That is, the overtube base 2 is fixed so that the relative position with the bed B, where a patient into which the overtube 1 is inserted lies, does not change during the treatment.

As shown in FIG. 2, the overtube base 2 includes an advance/retreat stopper 21, a wire stopper 22, a wire stopper drive part 23, a mounting part 7, a position sensor or distance sensor 8, and a controller (control part) 9.

The advance/retreat stopper 21 is a convex member formed on the overtube base 2, and engages with the advance/retreat stopper engaging part 63 of the operation part main body 61. Thereby, the operation part main body 61 and the overtube 1 cannot advance or retreat in the longitudinal axis direction.

Since the relative position of the overtube base 2 with the bed B where the patient under treatment lies is fixed, the overtube 1, which is fixed so as not to advance or retract in the longitudinal axis direction by the advance/retreat stopper 21 formed on the overtube base 2, is also fixed so as not to advance or retract in the longitudinal axis direction with respect to the bed B where the patient under treatment lies.

By releasing the engagement between the advance/retreat stopper 21 and the advance/retreat stopper engaging part 63 of the operation part main body 61, the operation part main body 61 and the overtube 1 can advance and retreat in the longitudinal axis direction.

The wire stopper (stopper) 22 is a convex member formed on the overtube base 2 and engages with the wire stopper engaging part 64 of the wire operation part 62.

The wire stopper 22 is arranged so that the relative position with the advance/retreat stopper 21 is constant. Thereby, the relative position between the wire operation part 62 engaged with the wire stopper 22 and the operation part main body 61 engaged with the advance/retreat stopper 21 is kept constant.

The wire stopper 22 is configured to be movable to either the first position or the second position by the wire stopper drive part 23.

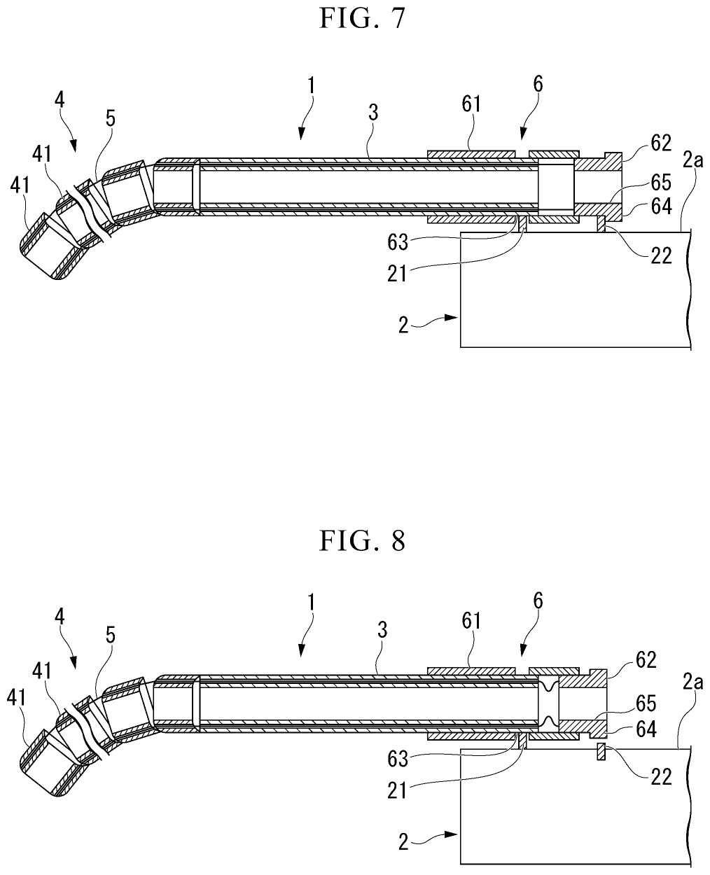

FIG. 7 is a cross-sectional view of the overtube device 1 when the wire stopper 22 is provided at the first position. The first position is a position protruding from the operation part mounting surface 2a of the overtube base 2 in the direction of the operation part mounting position.

On the other hand, FIG. 8 is a cross-sectional view of the overtube device 1 when the wire stopper 22 is provided at the second position. As shown in FIGS. 7 and 8, the second position is a position protruding from the operation part mounting surface 2a of the overtube base 2 in the direction of the operation part mounting position, and is a position where the protruding amount from the operation part mounting surface 2a is smaller than the protruding amount at the first position. The second position may be at a position that does not protrude from the operation part mounting surface 2a of the overtube base 2 in the direction of the operation part mounting position.

As shown in FIG. 7, when the wire stopper 22 is disposed at the first position, as shown in FIG. 7, the advance/retreat stopper 21 engages with the advance/retreat stopper engaging part 63, and the wire stopper 22 engages with the wire stopper engaging part 64. Thereby, the operation part 6 is mounted (attached) to the overtube base 2.

As shown in FIG. 8, when the wire stopper 22 is arranged at the second position, the wire stopper 22 cannot engage with the wire stopper engaging part 64 as shown in FIG. 8. Therefore, when the wire stopper 22 that engages with the wire stopper engaging part 64 is moved to the second position, the engagement between the wire stopper 22 and the wire stopper engaging part 64 is released.

As shown in FIG. 7, when the wire stopper 22 is disposed at the first position, the wire operation part 62 pulls the wire 5 to increase the tension of the wire 5, and the shape lock function of the bending part 4 is activated.

As shown in FIG. 8, when the wire stopper 22 is disposed at the second position, the wire operation part 62 does not pull the wire 5, and the shape lock function of the bending part 4 is invalidated.

By releasing the engagement between the wire stopper 22 and the wire stopper engaging part 64, the wire operating part 62 including the wire stopper engaging part 64 can be moved relative to the operating part main body 61.

The wire stopper drive part (stopper drive part) 23 moves the wire stopper from the first position to the second position, or from the second position to the first position. The wire stopper drive part 23 of the present embodiment is configured by a motor, a feed screw, and the like, and is controlled by the controller 9 described later.

The mounting part 7 is provided on the overtube base 2 for controlling the mounted treatment tool 400 (medical device). The mounting part 7 performs two types of control, that is, the advance/retreat operation of the treatment instrument 400 and the wire operation of the treatment instrument 400.

The mounting part 7 is provided on the overtube base 2 so as to be able to advance and retract in a linear direction, and can be advanced and retracted by a motor unit or the like provided on the overtube base 2. The linear direction coincides with the longitudinal axis direction of the overtube 1 when the operation part 6 is mounted (attached) on the overtube base 2. That is, the mounting part 7 moves the entire treatment instrument 400 relative to the carriage D in the longitudinal axis direction. As a result, the insertion part of the treatment instrument 400 is guided by the overtube 1 to advance and retract.

The mounting part 7 controls the wire operation of the mounted treatment tool 400. The treatment instrument 400 is provided with an operation wire for operating the treatment part 401 such as a grasping mechanism provided at the distal end, and the operation wire extends from the treatment part 401 to the proximal end part of the treatment instrument 400. By mounting the proximal end part of the treatment tool 400 on the mounting part 7, the proximal end part of the operation wire is connected to an operation wire drive part such as a pulley inside the mounting part 7. The operation wire drive part controls the operation wire to operate the operation of the treatment part 401.

The position sensor or distance sensor 8 is a sensor that determines whether the mounting part 7 is in a predetermined position range. As shown in FIGS. 1 and 2, the position sensor or the distance sensor 8 is arranged on more proximal end side than the mounting part 7 in the longitudinal axis direction of the overtube 1 when the operation part 6 is mounted (attached) on the overtube base 2. Therefore, when the mounting part 7 moves (retreats) in the direction away from the overtube 1 in the longitudinal axis direction, the position sensor or the distance sensor 8 can detect that the distance d between the mounting part 7 and the position sensor or distance sensor 8 is shortened. That is, the position sensor or the distance sensor 8 can detect the distance d and detect the position of the proximal end part of the treatment instrument 400 mounted on the mounting part 7.

In the present embodiment, the position sensor or distance sensor 8 is a sensor that electrically detects a distance using infrared rays or the like. Moreover, it may be a sensor that detects a distance using an ultrasonic wave or a laser.

Note that the position sensor or the distance sensor 8 may be a position sensor that can detect the position of the proximal end part of the treatment instrument 400 incorporating a magnetic source, for example.

The controller 9 controls the operation of the overtube base 2. The controller 9 controls the operation wire relative to the mounting part 7. In addition, the controller 9 controls the wire stopper drive part 23 to adjust the amount of protrusion of the wire stopper 22 from the operation part mounting surface 2a of the overtube base 2. The controller 9 receives the output of the position sensor or the distance sensor 8 and the output of the operation input part capable of operating the treatment instrument 400 provided on the carriage D, as input information. Since the controller 9 controls the operation wire for operating the treatment part 401 by itself, it is possible to grasp the curved shape of the joint of the treatment part 401.

Specific control contents will be described in the description of the operation of the overtube device 100 described later.

The controller 9 is a control device including a CPU (Central Processing Unit) and a memory, and the CPU generates an output (command) signal from an input (command) signal based on a program stored in the memory.

The controller 9 is not limited to the control device. The controller 9 may be a control circuit configured by a logic circuit.

The multi-lumen tube 200 is made of a flexible material such as silicone. As shown in FIGS. 3 and 4, the multi-lumen tube 200 is inserted through the internal space of the flexible tube part 3 and the bending piece 41.

In the multi-lumen tube 200, a first lumen 201, through which an observation means such as an endoscope 300 is inserted, and two second lumens 202, which has an inner diameter smaller than that of the first lumen 201 and through which the treatment instrument 400 is inserted, are provided over the entire length. The first lumen 201 and the second lumen 202 are both open at the proximal end side and the distal end side.

As shown in FIGS. 1, 3, and 4, the endoscope 300 is inserted into the first lumen 201 of the multi-lumen tube 200, and an imaging part 301 provided at the distal end of the insertion part of the endoscope 300 protrudes from the distal end of the overtube 1.

As shown in FIGS. 1, 3, and 4, the treatment tool 400 is inserted into the second lumen 202 of the multi-lumen tube 200, and a treatment part 401 such as a grip provided at the distal end of the insertion part of the treatment tool 400 protrudes from the distal end of the overtube 1.

The affected part is treated by the imaging part 301 of the endoscope 300 protruding from the distal end of the overtube 1 and the treatment part 401 of the treatment instrument 400.

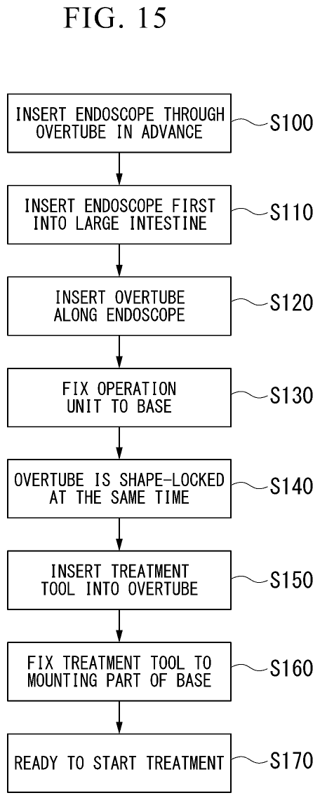

Next, the operation of the overtube device 100 will be described with reference to FIGS. 9 to 14. Here, the operation of inserting the overtube 1 into the large intestine L of the patient will be described. FIG. 15 shows a preparation procedure (S100 to S170) for starting treatment of an affected part using the overtube device 100.

First, the multi-lumen tube 200 is inserted through the overtube 1, and the endoscope 300 is inserted through the multi-lumen tube 200 of the overtube 1 (preparation procedure S100).

Next, as shown in FIG. 9, the endoscope 300 having an active bending part at the distal end is inserted into the large intestine L of the patient (preparation procedure S110). The operator inserts the distal end of the insertion part of the endoscope 300 to the treatment affected part of the large intestine L while actively bending the active bending part according to the curved shape of the large intestine L.

Next, as shown in FIG. 10, the operator inserts the multi-lumen tube 200 and the overtube 1 along the endoscope 300 (preparation procedure S120). Since the tension of the wire 5 does not exist, the shape lock function of the bending part 4 is invalidated, and the bending part 4 of the overtube 1 is inserted while being bent along the curved shape of the endoscope 300.

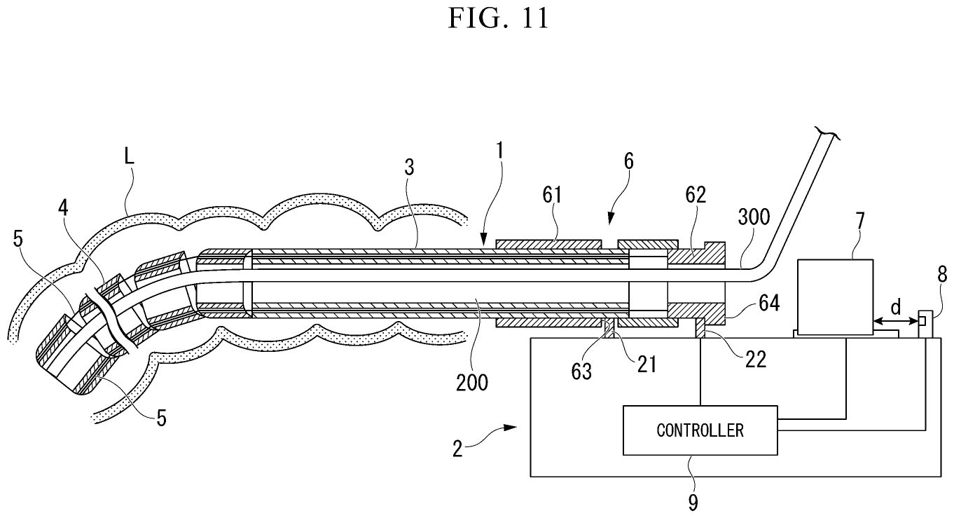

Next, as shown in FIG. 11, the operator mounts the operation part 6 on the overtube base 2 (preparation procedure S130). When mounting (attaching) the operation part 6 to the overtube base 2, the operator engages the advance/retreat stopper 21 with the advance/retreat stopper engaging part 63. At the same time, the operator engages the wire stopper 22 with the wire stopper engaging part 64 while pulling the wire operating part 62 toward the proximal end.

At the same time that the overtube 1 is fixed so as not to advance or retract in the longitudinal axis direction, the shape lock function is activated (preparation procedure S140).

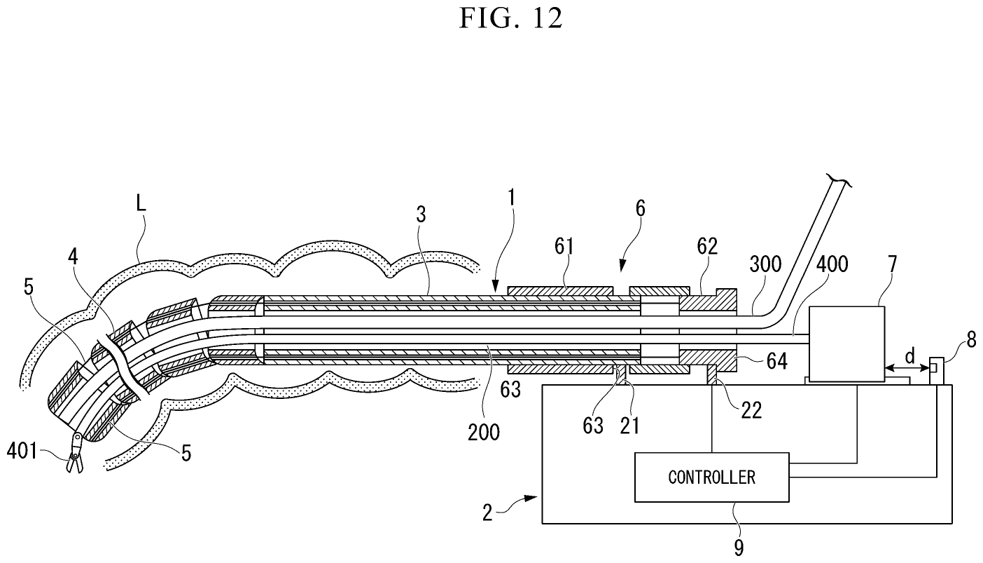

Next, as shown in FIG. 12, the operator inserts the treatment tool 400 into the second lumen 202 of the multi-lumen tube 200 (preparation procedure S150). The overtube 1 whose shape is temporarily fixed can more reliably guide the distal end of the treatment instrument 400 to the distal end of the overtube 1.

The proximal end of the treatment tool 400 is mounted on the mounting part 7 (preparation procedure S160). The controller 9 moves the treatment tool 400 in the longitudinal axis direction relative to the carriage D by operating the mounting part 7 based on input information related to the treatment tool 400 such as the operation input part. The insertion part of the treatment tool 400 is guided by the second lumen 202 of the multi-lumen tube 200 to advance or retreat. In addition, the controller 9 operates the treatment part 401 by controlling an operation wire from input information regarding the treatment part 401.

With the procedure so far, the preparation for starting the treatment of the affected part using the overtube device 100 is completed (preparation procedure S170).

The operator treats the affected part with the imaging part 301 of the endoscope 300 protruding from the distal end of the overtube 1 and the treatment part 401 of the treatment tool 400. The overtube 1 whose shape is temporarily fixed can stably arrange the endoscope 300 and the treatment tool 400 when the affected part of the large intestine L is treated.

However, in the conventional overtube device, when the operator greatly retracts the treatment instrument, the treatment part may come into contact with the overtube whose shape is temporarily fixed when the treatment part is pulled into the second lumen in the overtube, thereby damaging the overtube.

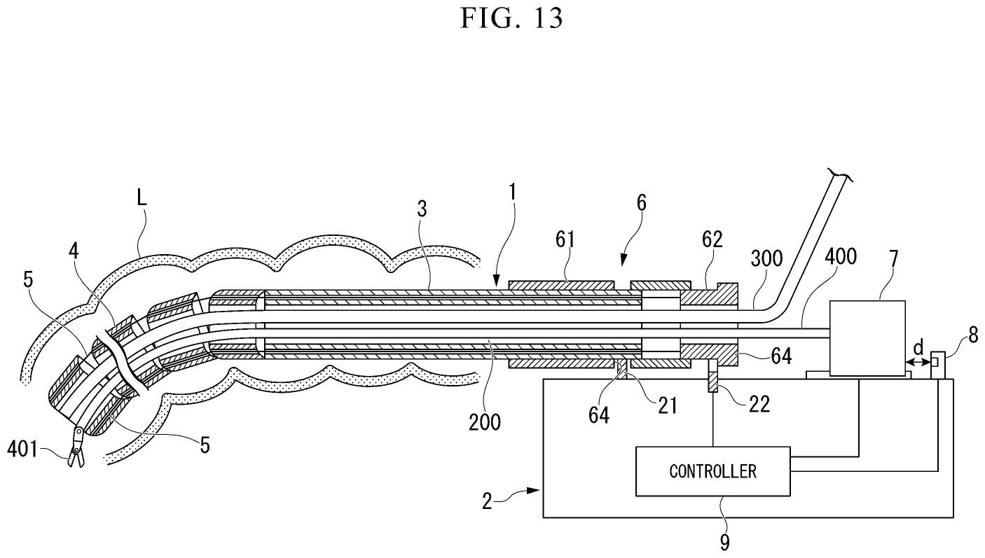

In the overtube device 100 of the present embodiment, as shown in FIG. 13, when the position sensor or distance sensor 8 detects that the distance d between the mounting part 7 and the position sensor or distance sensor 8 is equal to or less than a predetermined threshold value, the position sensor or distance sensor 8 outputs the detection result to the controller 9.

The threshold value is set so that the detection is performed in a state immediately before the mounting part 7 is retracted and the treatment part 401 is pulled into the overtube 1.

The controller 9 can grasp the curved shape of the joint of the treatment part 401, and predicts whether the treatment part 401 contacts the overtube 1 when the mounting part 7 is further retracted and pulled into the overtube 1.

For example, as shown in FIG. 13, when the treatment part 401 is bent with respect to the axial direction of the treatment instrument 400, there is a high possibility that the treatment part 401 comes into contact with the overtube 1 and is caught. The greater the bending of the treatment part 401 with respect to the axial direction, the higher the possibility that the treatment part 401 comes into contact with the overtube 1 and is caught.

When receiving a signal indicating that the distance d is equal to or less than a predetermined threshold value from the position sensor or the distance sensor 8 and determining that there is a high possibility that the overtube 1 is damaged based on the bending state of the joint of the treatment part 401, the controller 9 generates a control signal for moving the wire stopper 22 from the first position to the second position, to output to the wire stopper drive part 23. The wire stopper drive part 23 that has received the control signal moves the wire stopper 22 from the first position to the second position.

When receiving the signal indicating that the distance d is equal to or less than a predetermined threshold value from the position sensor or the distance sensor 8, regardless of the bending state of the joint of the treatment part 401, the controller 9 may generate a control signal for moving the wire stopper 22 from the first position to the second position to output to the wire stopper drive part 23. Control of the controller 9 is simplified.

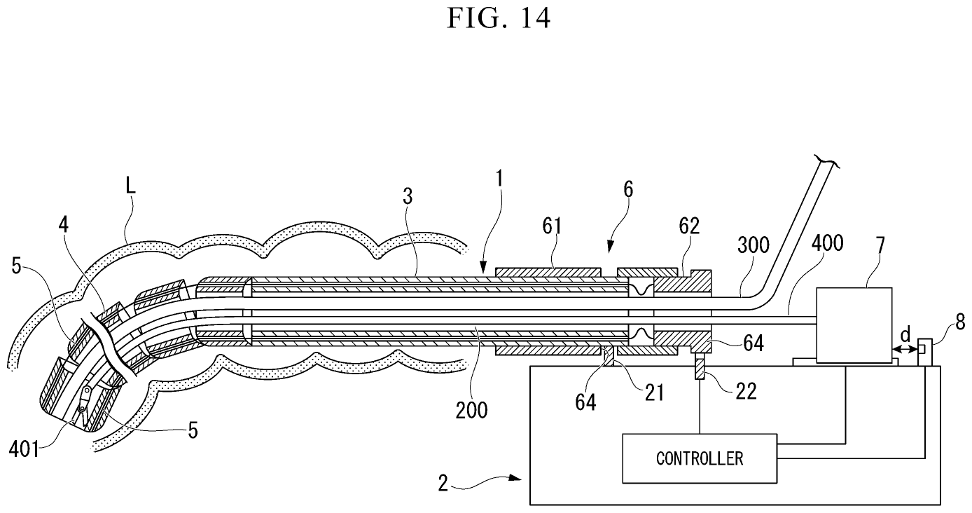

By the control of the controller 9 described above, the wire stopper 22 is moved to the second position as shown in FIG. 14, and the wire operation part 62 does not pull the wire 5 and the shape lock function of the bending part 4 is invalidated. In the bending part 4 in which the shape lock function is invalidated, the holding of the curved shape of the bending part 4 is released. When the treatment part 401 inclined with respect to the axial direction is drawn into the overtube 1, the bending part 4 can be bent in the same direction as the direction in which the treatment part 401 is inclined, as shown in FIG. 14. As a result, the possibility that the treatment part 401 contacts the overtube 1 is reduced. With this control, the overtube device 100 can prevent the overtube 1 from being damaged.

When the overtube 1 is advanced or retracted in order to change the affected part to be treated, the operator pulls up the operation section 6 of the overtube 1 from the overtube base 2 and separates them. By this operation, the engagement between the advance/retreat stopper 21 and the advance/retreat stopper engaging part 63 is released, and the engagement between the wire stopper 22 and the wire stopper engaging part 64 is also released. That is, when the overtube 1 is advanced or retracted, the shape lock function of the bending part 4 is automatically invalidated.

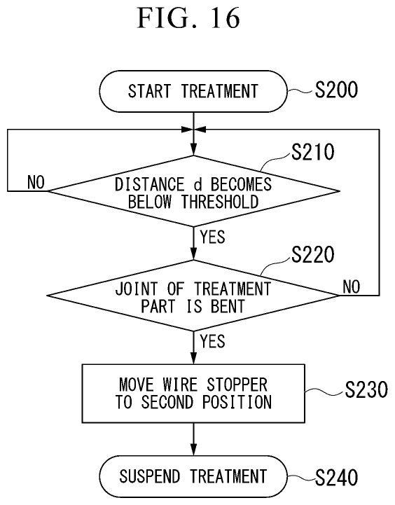

Next, the control flow of the controller 9 after the treatment of the affected part by the overtube device 100 will be described with reference to the flowchart of FIG. 16.

After the treatment is started (step S200), the controller 9 periodically observes a signal indicating that the distance d from the position sensor or the distance sensor 8 is equal to or less than a predetermined threshold value (step S210). When a signal indicating that the distance d is equal to or less than the predetermined threshold value is input, the controller 9 moves the control to S220.

In step S220, the controller 9 calculates whether the joint of the treatment part 401 is bent with respect to the axial direction from the control information of the treatment part 401 that the controller 9 has. When the joint of the treatment part 401 is bent with respect to the axial direction, the controller 9 shifts the control to step S230. If the joint of the treatment part 401 is not bent with respect to the axial direction, the controller 9 moves the control to step S210, and again observes a signal indicating that the distance d from the position sensor or the distance sensor 8 is equal to or less than a predetermined threshold value.

In step S230, the controller 9 outputs a control signal for moving the wire stopper 22 from the first position to the second position, to the wire stopper drive part 23. The wire stopper drive part 23 that has received the control signal moves the wire stopper 22 from the first position to the second position.

When the wire stopper 22 moves to the second position and the shape lock function of the bending part 4 is invalidated, the operator interrupts the treatment of the affected part (step S240). The operator again restarts the preparation procedure from S130 of the preparation procedure of the overtube device 100. The operator returns the wire stopper 22 from the second position to the first position before mounting (attaching) the operation part 6 to the overtube base 2.

In the control flow of the controller 9 described above, step S210 and step S220 may be controlled by switching their order. The controller 9 may perform control to move the wire stopper 22 from the first position to the second position when the distance d is equal to or less than a predetermined threshold value and the joint of the treatment part 401 is bent.

According to the overtube device 100 of the present embodiment, the shape lock function is invalidated based on the distance by which the treatment tool 400 retracts and the curved shape of the treatment part 401, and the overtube 1 can be prevented from being damaged. Even when the operator retracts the treatment instrument 400 before invalidating the shape lock function, the overtube device 100 invalidates the shape lock function.

Moreover, by providing the overtube device 100 with such a function, it is possible to reduce the operation burden on the operator during the treatment, and the operator can concentrate on the treatment.

As described above, although an embodiment of the present invention has been described in detail with reference to drawings, the concrete structure is not restricted to this embodiment. The design change or the like in the range which does not deviate from the summary of the present invention is included. In addition, the constituent elements shown in the above-described embodiment and the modified examples described below can be appropriately combined.

### Modification

For example, in the above embodiment, the curved shape of the treatment part 401 is calculated from the control information of the treatment part 401 that the controller 9 has. However, the manner of grasping the curved shape of the treatment part 401 is not limited to this. For example, an encoder may be provided on the joint of the treatment part 401, and the joint bending information acquired by the encoder may be output to the controller 9.

In the above embodiment, the treatment part 401 is a grasping mechanism. However, the treatment part 401 is not limited to this. For example, the treatment part 401 may be a knife having a joint. Based on the state of the joint of the treatment part 401, the controller 9 predicts whether the treatment part 401 contacts the overtube 1.

In the above embodiment, the retreat of the treatment instrument 400 is detected and the shape lock function is invalidated. However, the same processing may be performed on the endoscope 300 (medical device). In an endoscope having an active bending part at the distal end, the curved shape of the active bending part may be fixed. By detecting the retraction of the endoscope 300 and invalidating the shape lock function, it is possible to prevent the overtube from being damaged by the active bending part of the endoscope.

The bending part 4 of the above embodiment does not have a function to bend actively, but the aspect of the bending part 4 is not limited to this. For example, the overtube 1 may have an active bending part that bends actively at least at a part of the bending part 4. When having the active bending part, the overtube 1 may have an angle wire that bends the active bending part.

The multi-lumen tube 200 is inserted into the overtube 1 of the above embodiment, but a plurality of thimble lumen tubes may be used instead of the multi-lumen tube 200.

The advance/retreat stopper 21 of the above embodiment is a convex member, and the advance/retreat stopper engaging part 63 is a concave member. However, the modes of the advance/retreat stopper 21 and the advance/retreat stopper engaging part 63 are not limited to this. The advancing/retreating stopper 21 and the advancing/retreating stopper engaging part 63 may be any shape as long as they are engaged and the overtube base 2 can fix the overtube 1 so as not to advance and retreat in the longitudinal axis direction. For example, the advance/retreat stopper may be a concave member, and the advance/retreat stopper engaging part may be a convex member.

The wire stopper 22 of the above embodiment is a convex member, and the wire stopper engaging part 64 is also a convex member. However, the aspect of the wire stopper 22 and the wire stopper engaging part 64 is not limited to this. The wire stopper 22 and the wire stopper engaging part 64 may have any shape as long as they are engaged and the relative position between the wire operation part 62 and the operation part main body 61 can be kept constant. For example, the wire stopper may be a concave member and the wire stopper engaging part may be a convex member.

The number of wires 5 and wire lumens (31, 42) may be other than four. For example, by bringing the bending pieces 41 into close contact with more wires 5, the frictional force between the bending pieces 41 becomes higher. By increasing the frictional force between the bending pieces 41, the curved shape of the bending part 4 is more stably held when the shape lock function is activated.

In the above-described embodiment, whether or not the joint of the treatment part 401 is bent with respect to the axial direction is one condition for invalidating the shape lock function. However, the condition for invalidating the shape lock function is not limited to this. For example, by comparing the shape of the treatment part 401 with the inner diameter of the overtube 1 or the multi-lumen tube 200 into which the treatment part 401 is drawn, whether the treatment part 401 actually contacts the inside of the overtube 1 or the multi-lumen tube 200 and is caught may be calculated. The calculation result may be one condition for invalidating the shape lock function.

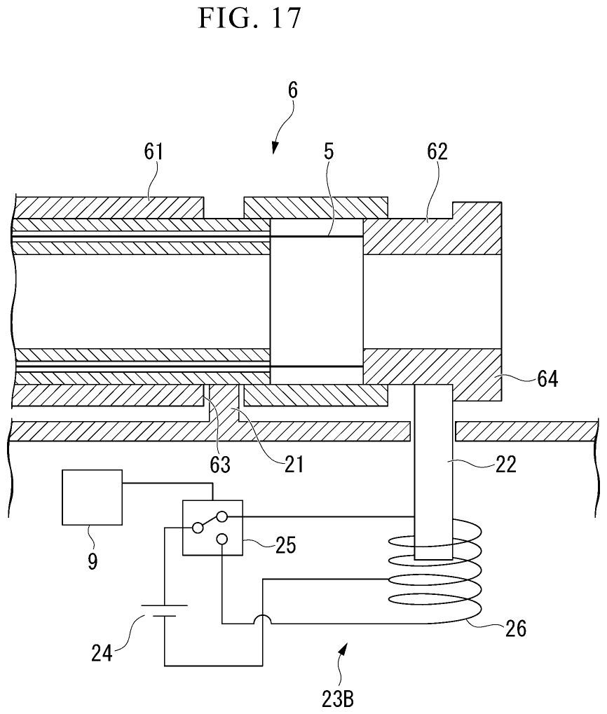

Moreover, in the above embodiment, the wire stopper drive part 23 is driven with the motor, but the driving mode of the wire stopper 22 by the wire stopper drive part 23 is not limited to this. For example, a wire stopper drive part 23B, which is a modification of the wire stopper drive part 23 shown in FIG. 17, includes a power source 24, a relay 25, and a solenoid 26. The controller 9 controls the relay to form a closed circuit including the power source 24, the relay 25, and the solenoid 26, so that the solenoid 26 drives the wire stopper 22 provided inside the solenoid 26.

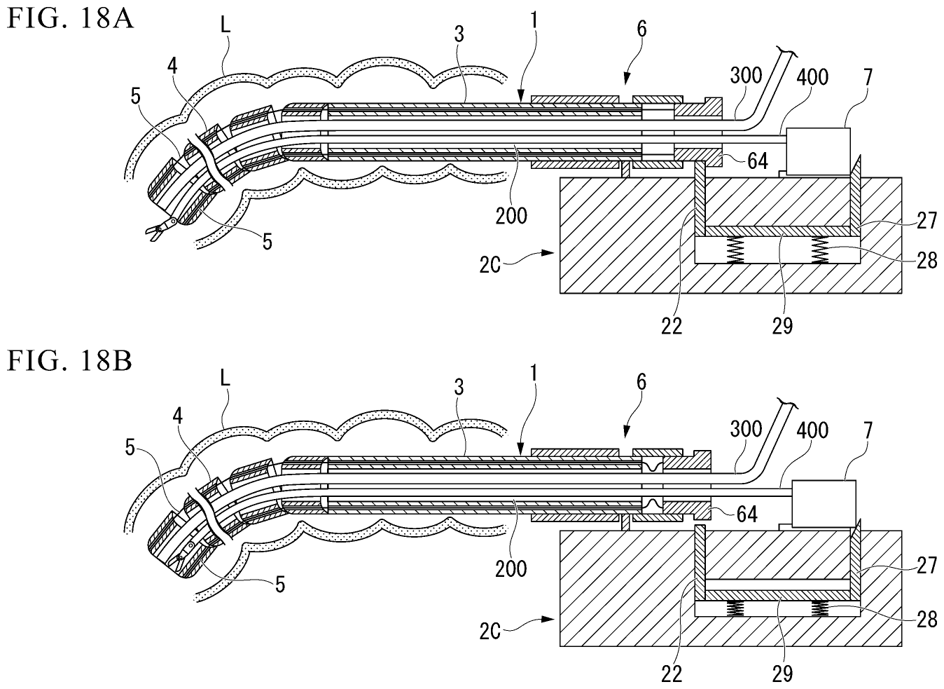

In the above embodiment, the detection of the retraction of the mounting part 7 and the driving of the wire stopper 22 are performed by electrical control. However, the manner of detecting the retraction of the mounting part 7 and the driving of the wire stopper 22 are not limited to this. For example, the detection of the retraction of the mounting part 7 and the driving of the wire stopper 22 may be performed by mechanical control. An overtube base 2C, which is a modification of the overtube base 2 shown in FIG. 18, has a retraction detection part 27, a spring 28, and a link 29 instead of the position sensor or distance sensor 8 and the controller 9. The wire stopper 22 and the retraction detection part 27 are connected by a link 29. The link 29 is urged by a spring 28 so that the wire stopper 22 and the retraction detection part 27 protrude from the operation part mounting surface 2a of the overtube base 2 in a direction (projection direction) toward the operation part mounting position.

FIG. 18 shows a configuration in which the operation part 6 is mounted (attached) on the overtube base 2C.

As shown in FIG. 18A, when the mounting part 7 does not retract to a predetermined position, the wire stopper 22 is biased by the spring 28 so as to protrude in the protruding direction. The wire stopper 22 is biased so as to protrude in the protruding direction, thereby engaging the wire stopper engaging part 64. In this state, the shape lock function of the bending part 4 is activated.

When the mounting part 7 retracts to a predetermined position, as shown in FIG. 18B, the retreat detecting part 27 engages with the mounting part 7 and moves in the direction opposite to the protruding direction. As a result, the wire stopper 22 connected by the link 29 also moves in the direction opposite to the protruding direction, the engagement between the wire stopper 22 and the wire stopper engaging part 64 is released, and the shape lock function of the bending part 4 is invalidated.

Thus, the detection of the backward movement of the mounting part 7 and the driving of the wire stopper 22 may be performed by mechanical control.

What is claimed is:

1. An overtube device comprising:
a tube body having a bending part that is curvable on a distal end side, and a main body part that is continuous with the bending part and extends to a proximal end side;
a wire having a distal end part fixed to the bending part, and a proximal end part positioned on the proximal end side of the main body part and extending along a longitudinal axis of the main body part;
an operation part that is provided on the proximal end side of the main body, is attached to the proximal end part of the wire, and is configured to be capable of pulling the wire to the proximal end side of the wire; and
an overtube base having a stopper configured to fix the tube body in a longitudinal axis direction so as not to advance and retract, and to engage with the operation part in a state where the wire is pulled toward the proximal end side of the wire so as to hold a curved shape of the bending part,
wherein, when a medical device inserted into the tube body is retracted, an engagement between the operation part and the stopper is released, and holding of the curved shape of the bending part is also released at the time when the medical device is retracted.

2. The overtube device according to claim 1, wherein
the operation part includes:
an operation part main body; and
a wire operation part that is held so as to be relatively movable with respect to the operation part main body and to which the proximal end part of the wire is attached,
wherein, by moving the wire operation part relative to the operation part main body, the wire is pulled toward the proximal end side of the wire.

3. The overtube device according to claim 2, wherein
the operation part main body includes an advance/retreat stopper engaging part,
the wire operation part includes a wire stopper engaging part,
the stopper of the overtube base includes an advance/retreat stopper configured to engage with the advance/retreat stopper engaging part, and a wire stopper configured to engage with the wire operating part while keeping a relative position with the advance/retreat stopper constant, and
the advance/retreat stopper engages with the advance/retreat stopper engaging part and the wire stopper engages with the wire stopper engaging part, so that the operation part is mounted on the overtube base.

4. The overtube device according to claim 3, wherein
the wire stopper is configured to be movable to either a first position or a second position,
when the wire stopper that engages with the wire stopper engaging part is disposed at the first position, the operation part pulls the wire toward the proximal end side of the wire, and the curved shape of the bending part is held, and
when the wire stopper that engages with the wire stopper engaging part is disposed at the second position, the operation part does not pull the wire toward the proximal end part of the wire, and the curved shape of the bending part is released.

5. The overtube device according to claim 1, wherein
the overtube base includes:
a sensor configured to detect a position of a proximal end of the medical device;
a stopper drive part configured to operate the stopper; and
a controller configured to determine whether or not the medical device is retracted based on a detection result by the sensor, and to operate the stopper drive part so that the engagement between the operation part and the stopper is released when it is determined that the medical device is retracted, and
by release of the engagement between the operation part and the stopper by the stopper drive part, holding of the curved shape of the bending part is released when the medical device is retracted.

6. The overtube device according to claim 1, wherein
the overtube base includes:
a sensor configured to detect a position of a proximal end of the medical device;
a stopper drive part configured to operate the stopper; and
a processor configured to determine whether or not the medical device is retracted based on a detection result by the sensor, and to generate a command to operate the stopper drive part so that the engagement between the operation part and the stopper is released when it is determined that the medical device is retracted, and
by release of the engagement between the operation part and the stopper by the stopper drive part based on the command from the processor, holding of the curved shape of the bending part is released when the medical device is retracted.

7. The overtube device according to claim 6, wherein
the processor controls an operation of bending a distal end part of the medical device, and
when it is determined that the medical device is retracted and when a curved shape of the distal end part is a shape that contacts the tube body, the processor controls the engagement between the operation part and the stopper to release the holding of the curved shape of the bending part.

8. The overtube device according to claim 1, wherein
the overtube base includes:
a retraction detection part; and
a link that connects the stopper to the retreat detection part, and
a proximal end part of the medical device engages with the retraction detection part and the stopper is operated in conjunction, so that the engagement between the operation part and the stopper is released.

9. The overtube device according to claim 1, wherein
the wire is a plurality of wires, and
the stopper of the overtube base engages with the operation part in a state where the plurality of wires are simultaneously pulled toward a proximal end side of the plurality of wires.
